(19) United States
(12) Patent Application Publication
(10) Pub. No.: US 2014/0155795 A1
(43) Pub. Date: Jun. 5, 2014

(54) Sports Training Devices and Methods for Using Same

(71) Applicant: Benjamin Evenden, Dallas, TX (US)

(72) Inventor: Benjamin Evenden, Dallas, TX (US)

(21) Appl. No.: 13/706,147

(22) Filed: Dec. 5, 2012

(51) Int. Cl. A61F 5/01

(52) U.S. Cl. CPC A61F 5/012; A61F 5/013
USPC 602/13

(57) ABSTRACT

A sports training device may be formed as a cuff that may be wrapped around a user's elbow. The cuff may be formed of a flexible material, such as neoprene, and may include a fastening mechanism, such as a hook-and-loop fastener, to close the cuff around the user's elbow. The sports training device may include an adjustable air bladder having a pump and a release valve. The adjustable air bladder may be positioned around the user's elbow and adjusted using the pump and valve mechanism such that air may be distributed within the sports training device. Accordingly, use of the device may avoid degrees of bending with respect to the user's elbow, such as when swinging a tennis racket.

### FIELD OF THE DISCLOSURE

The present disclosure generally relates to sports training devices, and more particularly to sports training devices for improvement of swinging motion and methods for using same.

### BACKGROUND

When someone is learning to play a sport, such as tennis, one of the more difficult things is to keep the user's arm straight at the appropriate times when he/she swings the tennis racket. A user might wear a brace to keep his/her arm straight, but the repetitive motion of swinging may cause pain in the user's arm or elbow over time. Further, different degrees of bending may still occur.

### SUMMARY

Embodiments of the present disclosure may provide a sports training device worn around a user's elbow, the sports training device comprising a flexible material that wraps circumferentially around the user's elbow, an adjustable air bladder mounted to the flexible material and positioned at the elbow pit region of the user's elbow, a pump coupled to the adjustable air bladder, wherein the pump inflates the adjustable air bladder to restrict movement of the user's elbow at a constant variable. The sports training device also may include a release valve to reduce the inflation of the adjustable air bladder. The sports training device may further comprise a pocket integrally attached to the flexible material, the pocket housing the adjustable air bladder and the pump. The pocket may be releasably attached to the flexible material and house the adjustable air bladder and the pump. The flexible material may be selected from the group comprising neoprene, silicone elastomer, polyester, nylon, taslon, and combinations of the same. The flexible material also may comprise elastic. The sports training device may comprise a fastening mechanism to fasten one end of the flexible material to the other end of the flexible material around the user's elbow. This fastening mechanism may be a fabric hook-and-loop fastener. The sports training device may also include a release valve to reduce air pressure within the adjustable air bladder and loosen the position of the sports training device around the user's elbow. The adjustable air bladder may be coupled to the pump through a connector extending from the pump to the adjustable air bladder. The sports training device also may include a hole formed within the flexible material to receive the user's olecranon. The region of the flexible material around the hole may be curved to hug the user's bicep when the sports training device is secured around the user's elbow.

Other embodiments of the present disclosure may provide a method for using a sports training device, the method comprising wrapping the sports training device around a user's elbow region, the sports training device comprising a flexible material, an adjustable air bladder and a pump, wherein the sports training device may be wrapped to position the adjustable air bladder at the elbow pit of the user's elbow region, and applying pressure to the pump to inflate the adjustable air bladder, wherein the user's elbow region may be restricted in its movement when swinging. The method also may comprise using a release valve coupled to the adjustable air bladder to selectively reduce pressure within the adjustable air bladder.

Additional embodiments of the present disclosure may provide a sports swing teaching aid comprising a flexible material circumferentially disposed around a user's elbow in a cuff formation, the flexible material having a pocket positioned in the elbow pit region of the user's elbow that receives an adjustable air bladder, and a pump and valve mechanism coupled with the adjustable air bladder to selectively distribute air within the adjustable air bladder and limit degrees of bending of the user's elbow. The pocket may be integrally formed with the flexible material. The pocket also may be releasably attached to the flexible material. The sports swing teaching aid also may include a fastening mechanism to fasten one end of the flexible material to the other end of the flexible material around the user's elbow. The adjustable air bladder may be coupled to the pump through a connector extending from the pump to the adjustable air bladder. The sports swing teaching aid also may include a hole formed within the flexible material to receive the user's olecranon.

### BRIEF DESCRIPTION OF THE DRAWINGS

For a more complete understanding of this disclosure, reference is now made to the following description, taken in conjunction with the accompanying drawings, in which:

FIG. 1 depicts an interior view of a sports training device according to an embodiment of the present disclosure;

FIG. 2 depicts an exterior view of the sports training device of FIG. 1 according to an embodiment of the present disclosure;

FIG. 3 depicts an interior view of another sports training device according to an embodiment of the present disclosure;

FIG. 4 depicts a sports training device when worn by a user according to an embodiment of the present disclosure; and

FIG. 5 depicts a perspective view of a sports training device according to an embodiment of the present disclosure.

### DETAILED DESCRIPTION

A sports training device according to embodiments of the present disclosure may be formed as a cuff that may be wrapped around a user's elbow. The cuff may be formed of a flexible material, such as neoprene, and may include a fastening mechanism, such as a hook-and-loop fastener, to close the cuff around the user's elbow according to embodiments of the present disclosure. The sports training device may include an adjustable air bladder having a pump and a release valve. The adjustable air bladder may be positioned around the user's elbow and adjusted using the pump and valve mechanism such that air may be distributed within the sports training device. Accordingly, use of the device may avoid degrees of bending with respect to the user's elbow, such as when swinging a tennis racket.

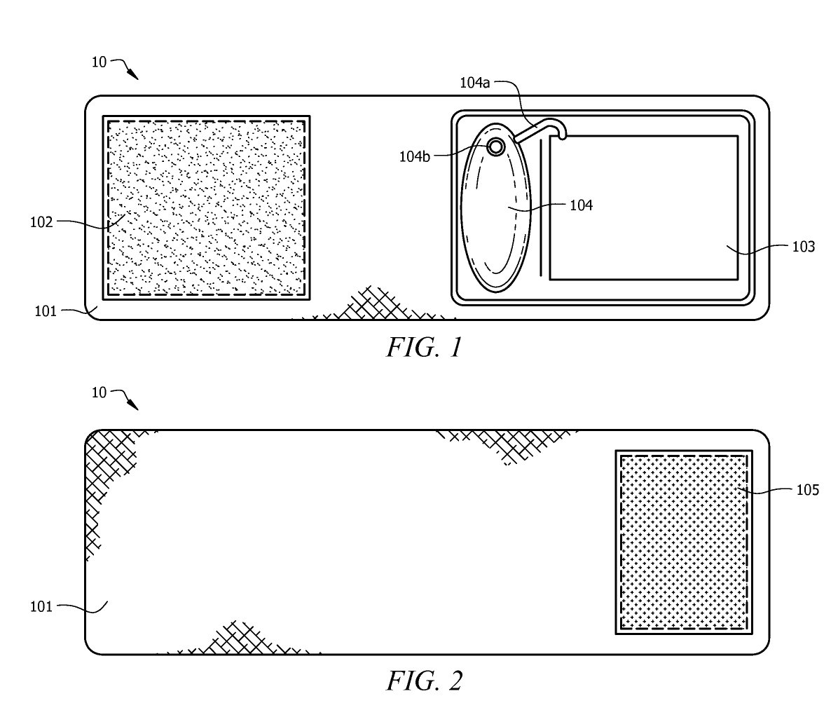

FIG. 1 depicts an interior view of sports training device 10 according to an embodiment of the present disclosure. In an embodiment of the present disclosure, sports training device 10 may be comprised of flexible material 101, fastening mechanism 102, air bladder 103 and pump 104. It should be appreciated that more or fewer components may be included as part of sports training device 10 without departing from the present disclosure. Each of these components is described in more detail below.

Flexible material 101 may be formed of a flexible fabric or material that may be comfortably wrapped around a user's elbow, such as in a cuff formation, and a user may wear over an extended period of time when playing a sport, such as tennis, baseball or golf. Fabrics or materials forming flexible material 101 may include but are not limited to neoprene, silicone elastomer, polyester, nylon, taslon, and combinations thereof. In some embodiments of the present disclosure, flexible material 101 may incorporate a stretchy material, such as elastic, so that sports training device 10 may better conform to or be secured around the user's elbow. It should be appreciated that flexible material 101 may be formed as a strip wherein the ends are squared off according to embodiments of the present disclosure. In other embodiments of the present disclosure, the ends of flexible material 101 may be curved or otherwise shaped to accommodate whatever fastening mechanism that may be employed to secure sports training device 10 around a user's elbow.

Fastening mechanism 102 may comprise a fabric hook-and-loop fastener, such as Velcro, wherein fastening mechanism 102 may be joined with fastening mechanism 105 (depicted in FIG. 2 as part of the exterior view of sports training device 10 of FIG. 1) with a first fastening mechanism 102 or 105 comprising one or more fabric strips with tiny hooks and the second fastening mechanism comprising one or more fabric strips with loops to receive the hooks from the first fastening mechanism so as to fasten/bind opposing ends of flexible material 101 together around the wearer's elbow. However, it should be appreciated that other mechanisms for fastening may be employed without departing from the present disclosure. In another embodiment of the present disclosure, flexible material 101 may be formed as a closed loop wherein no separate fastening mechanism may be employed insofar as flexible material 101 may include elastic or another material that may otherwise secure sports training device 10 around a user's elbow.

Adjustable air bladder 103 may be incorporated as part of sports training device 10 in embodiments of the present disclosure. In an embodiment of the present disclosure, adjustable air bladder 103 may be coupled to pump 104 through connector 104a extending from pump 104 to adjustable air bladder 103 such that a user may selectively inflate or deflate adjustable air bladder 103 using pump 104. A user (or his/her trainer or coach) may depress pump 104 one or more times to slowly increase the air pressure within adjustable air bladder 103, thereby further restricting movement of the user's elbow (i.e., reducing the degree of bend in the user's elbow). When the user follows through on his/her swing while wearing sports training device 10, as the amount of air within air bladder 103 increases, the user's elbow may be better held in an extended range of motion position. Even as air is forced into air bladder 103, sports training device 10 may place an amount of pressure on the user's elbow to restrict movement while minimizing or eliminating pain that the user might otherwise feel during a repetitive movement such as when swinging a tennis racket.

Release valve 104b may be included as part of sports training device 10 so that air pressure built up inside adjustable air bladder 103 may be released to loosen the hold of sports training device 10 around a user's elbow. Release valve 104b also may assist the user or trainer/coach to tell whether sports training device 10 is working properly. For example, a user may place his/her hand in front of release valve 104b and feel whether air is escaping from sports training device 10.

It should be appreciated that there may be embodiments of the present disclosure wherein the adjustable air bladder and/or the pump may be housed within a bag or pocket that is sewn or otherwise attached to flexible material 101 forming sports training device 10. This mechanism for housing the air bladder and/or pump may further streamline the design of a sports training device according to embodiments of the present disclosure. It should be appreciated that the bag or pocket may be resealable, such as through a zipper or other closure mechanism, so that a user may access the air bladder or pump for repair or other adjustment purposes without departing from the present disclosure. There also may be other embodiments of the present disclosure wherein the bag or pocket is unsealed or open (i.e., does not contain a separate closure mechanism). In a further embodiment of the present disclosure, the bag or pocket may be releasably attached to flexible material 101, such as through snaps or a hook-and-loop fastener, such that the entire bag or pocket may be detached from flexible material 101 and replaced with another bag or pocket without departing from the present disclosure. This may be employed in instances where the air bladder and/or pump may be defective and it may be more efficient to replace the bag or pocket as well as its contents at one time. The bag or pocket may be formed of the same material as flexible material 101 according to embodiments of the present disclosure, but in other embodiments of the present disclosure, the bag or pocket may be formed of a stronger material to support the air bladder and/or pump such that they do not rip the bag or pocket open when sports training device 10 is fastened around a user's elbow and may be used for an extended period of time without damage.

FIG. 1 depicts adjustable air bladder 103 on an end of flexible material 101 opposite fastening mechanism 102; however, it should be appreciated that adjustable air bladder 103 may be selectively positioned toward the center of sports training device 10 and closer in proximity to fastening mechanism 102 without departing from the present disclosure. Similarly, pump 104 is depicted as being positioned on the left hand side of adjustable air bladder 103; however, it should be appreciated that pump 104 may be positioned in different places along the length of flexible material 101 with respect to adjustable air bladder 103 without departing from the present disclosure.

FIG. 2 depicts an exterior view of sports training device 10 of FIG. 1 according to an embodiment of the present disclosure. As previously discussed, fastening mechanism 105 may be positioned such that it may fasten or bind with fastening mechanism 102 positioned on the interior of sports training device 10 (see FIG. 1). While fastening mechanism 105 is depicted as a portion of a hook-and-loop fastener, it should be appreciated that fastening mechanism 105 may be substituted with another fastening mechanism without departing from the present disclosure. Further, while not explicitly depicted in FIG. 2, it should be appreciated that, when inflated, adjustable air bladder 103 may bulge and be visible in the exterior view of sports training device 10 depicted in FIG. 2. Similarly, depending on the size and shape of pump 104, pump 104 also may be visible in the exterior view of sports training device 10 according to embodiments of the present disclosure.

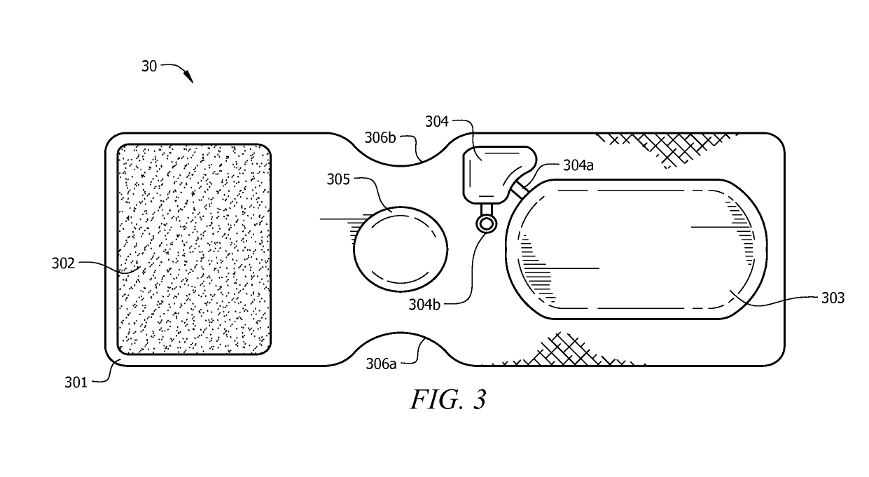

FIG. 3 depicts another interior view of sports training device 30 according to an embodiment of the present disclosure. In this embodiment of the present disclosure, sports training device 30 may include flexible material 301 (similar to flexible material 101 described with respect to FIG. 1) that may be wrapped around and secured to a user's elbow region. In this embodiment of the present disclosure, there may be hole 305 formed within flexible material 302 so that the user's olecranon (the bony prominence at the very tip of the elbow) may protrude out of flexible material 305 so as to make the olecranon less restricted and perhaps more comfortable for the user to wear sports training device 30. In addition, a portion of flexible material 301 surrounding hole 305 may be curved and curved portions 306a, 306b may hug the user's bicep when sports training device 30 is secured around the user's elbow thereby reducing the amount of twisting of the user's arm when he/she is wearing sports training device 30. Fastening mechanism 302 may be incorporated as part of sports training device 30 in a manner similar to that described with respect to fastening mechanism 102 of FIG. 1. Further, pump 304 may connect to adjustable air bladder 303 via connection 304a in a manner similar to that described with respect to FIG. 1. Pump 304 also may include release valve 304b; however, in this embodiment of the present disclosure, release valve 304b is coupled to pump 304 and is not formed as an integral part of pump 304 (as was release valve 104b as depicted in FIG. 1). While release valves have been depicted in different manners in FIGS. 1-2, it should be appreciated that there may be embodiments where there is no direct coupling or connection to pump 304. As such, release valve 304 may be positioned in another area of sports training device 30 without departing from the present disclosure.

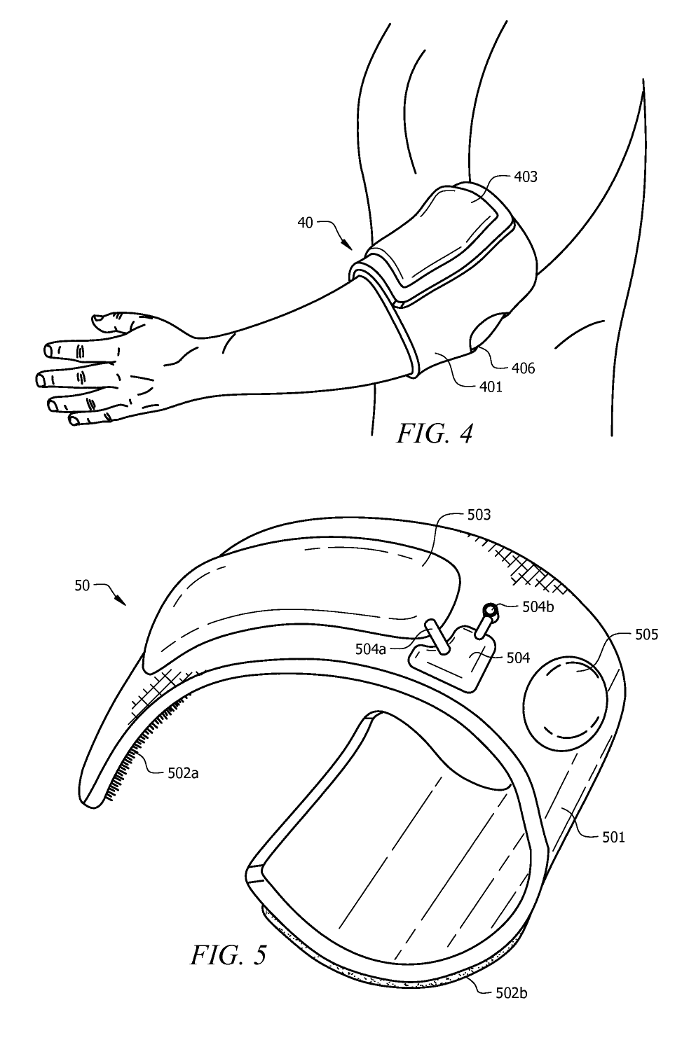

FIG. 4 depicts sports training device 40 when worn around a user's elbow according to an embodiment of the present disclosure. In this embodiment of the present disclosure, flexible material 401 may be secured around the user's elbow using a fastening mechanism, such as a fabric hook-and-loop fastener, as has been previously described. Hole 406 may be formed within flexible material 401 at so that the olecranon may protrude out of flexible material 401 during use. Adjustable air bladder 403 may then be positioned around the user's cubital fossa (also referred to as the elbow pit) in this embodiment of the present disclosure. However, as previously discussed, the positioning of adjustable air bladder 403 may be adjusted slightly with respect to the user's cubital fossa without departing from the present disclosure. In this embodiment of the present disclosure, the mechanism of fastening sports training device 40 around the user's elbow is not visible; however, it should be appreciated that fastening mechanisms as previously described with respect to FIGS. 1-2 may be employed without departing from the present disclosure. In other embodiments of the present disclosure, an elastic-type material may be incorporated into flexible material 401 such that the user may slide sports training device 40 onto his/her arm without the need for a fastening mechanism.

A user may hold a tennis racket in his/her hand while wearing sports training device 40. The amount of air contained within adjustable air bladder 403 may be adjusted at a constant variable so that when the user swings the racket, his/her elbow may remain straight through the swinging motion. As the user becomes more adept at swinging the racket, the amount of air pressure provided through adjustable air bladder 403 may be gradually adjusted or decreased so that the user may eventually swing the tennis racket properly without the need for use of sports training device 40.

FIG. 5 depicts a perspective view of sports training device 50 according to an embodiment of the present disclosure. In this embodiment of the present disclosure, adjustable air bladder 503 and pump 504 are depicted as being positioned away from the user's elbow when the user is wearing sports training device 50. As such, the user's trainer or coach may more easily access pump 504 connected to adjustable air bladder 504a to make changes to the pressure being applied to the user's elbow. Further, in this embodiment of the present disclosure, sports training device 50 may include release valve 504b that may be coupled to pump 504. However, as previously discussed, there may be embodiments wherein the release valve is formed as part of the pump or may not be directly coupled to the pump without departing from the present disclosure. Flexible material 501 is depicted in this embodiment of the present disclosure as a cuff having hole 505 to receive the user's olecranon when sports training device 50 is being worn. However, it should be appreciated that there may be other embodiments wherein no hole may be included. Flexible material 501 may include fastening mechanism 502a, 502b formed as a hook-and-loop fastener in this embodiment of the present disclosure. However, as previously discussed, other fastening mechanism may be employed to join the ends of flexible material 501 without departing from the present disclosure.

While embodiments of the present disclosure have been described as being directed to sports training devices for use in helping a user develop his/her tennis swing or follow-through, it should be appreciated that these sports training devices may be utilized with respect to other sports. For example, a user may employ a sports training device to assist with developing his/her swing in sports including but not limited to golf or baseball.

Further, it should be appreciated that sports training devices according to embodiments of the present disclosure may be formed so that a user may perform self-adjustments when practicing his/her swinging motion. However, there may be other embodiments of the present disclosure wherein the user is being trained or coached in the sport. As such, devices according to embodiments of the present disclosure may be formed so that the trainer/coach may apply pressure to the pump or access the release valve to adjust the amount of air contained within the adjustable air bladder while training or coaching the user.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A sports training device worn around a user's elbow, the sports training device comprising:
a flexible material that wraps circumferentially around the user's elbow;
an adjustable air bladder mounted to the flexible material and positioned at the elbow pit region of the user's elbow; and
a pump coupled to the adjustable air bladder, wherein the pump inflates the adjustable air bladder to restrict movement of the user's elbow at a constant variable.

2. The sports training device of claim 1 further comprising:
a release valve to reduce the inflation of the adjustable air bladder.

3. The sports training device of claim 1 further comprising:
a pocket integrally attached to the flexible material, the pocket housing the adjustable air bladder and the pump.

4. The sports training device of claim 1 further comprising:
a pocket releasably attached to the flexible material, the pocket housing the adjustable air bladder and the pump.

5. The sports training device of claim 1 wherein the flexible material is selected from the group comprising:
neoprene, silicone elastomer, polyester, nylon, taslon, and combinations of the same.

6. The sports training device of claim 5 wherein the flexible material further comprises elastic.

7. The sports training device of claim 1 further comprising:
a fastening mechanism to fasten one end of the flexible material to the other end of the flexible material around the user's elbow.

8. The sports training device of claim 7 wherein the fastening mechanism is a fabric hook-and-loop fastener.

9. The sports training device of claim 1 further comprising:
a release valve to reduce air pressure within the adjustable air bladder and loosen the position of the sports training device around the user's elbow.

10. The sports training device of claim 1 wherein the adjustable air bladder is coupled to the pump through a connector extending from the pump to the adjustable air bladder.

11. The sports training device of claim 1 further comprising:
a hole formed within the flexible material to receive the user's olecranon.

12. The sports training device of claim 12 wherein the region of the flexible material around the hole is curved to hug the user's bicep when the sports training device is secured around the user's elbow.

13. A method for using a sports training device, the method comprising:
wrapping the sports training device around a user's elbow region, the sports training device comprising a flexible material, an adjustable air bladder and a pump, wherein the sports training device is wrapped to position the adjustable air bladder at the elbow pit of the user's elbow region; and
applying pressure to the pump to inflate the adjustable air bladder, wherein the user's elbow region is restricted in its movement when swinging.

14. The method of claim 9, further comprising:
using a release valve coupled to the adjustable air bladder to selectively reduce pressure within the adjustable air bladder.

15. A sports swing teaching aid comprising:
a flexible material circumferentially disposed around a user's elbow in a cuff formation, the flexible material having a pocket positioned in the elbow pit region of the user's elbow that receives an adjustable air bladder; and
a pump and valve mechanism coupled with the adjustable air bladder to selectively distribute air within the adjustable air bladder and limit degrees of bending of the user's elbow.

16. The sports swing teaching aid of claim 15 wherein the pocket is integrally formed with the flexible material.

17. The sports swing teaching aid of claim 15 wherein the pocket is releasably attached to the flexible material.

18. The sports swing teaching aid of claim 15 further comprising:
a fastening mechanism to fasten one end of the flexible material to the other end of the flexible material around the user's elbow.

19. The sports swing teaching aid of claim 15 wherein the adjustable air bladder is coupled to the pump through a connector extending from the pump to the adjustable air bladder.

20. The sports training device of claim 15 further comprising:
a hole formed within the flexible material to receive the user's olecranon.
